(19) United States
(12) Patent Application Publication
(10) Pub. No.: US 2020/0135314 A1
(43) Pub. Date: Apr. 30, 2020

(54) PERSONALIZED MEDICATION MANAGEMENT AND ALERT SYSTEM AND METHOD

(71) Applicant: Pharmazam, LLC, Tampa, FL (US)

(72) Inventors: Scott Gostyla, Palm harbor, FL (US); Sean Carey, Tampa, FL (US); James Silvera, Tampa, FL (US); Taimour Langaee, Gainesville, FL (US); Anthony Holan, Tampa, FL (US); Nichols Holan, Tampa, FL (US)

(73) Assignee: Pharmazam, LLC, Tampa, FL (US)

(21) Appl. No.: 16/173,956

(22) Filed: Oct. 29, 2018

(51) Int. Cl. G16H 20/10; G06F 17/30

(52) U.S. Cl. CPC G16H 20/10; G06N 3/123; G06F 17/30283

(57) ABSTRACT

A personalized medication management and alert method and system is provided. The method includes, creating a user profile based at least upon received user genetic information, storing the user profile in memory on a computer, updating the user profile based on received user personal information, the received personal information including any medications currently used by the user, comparing, by a processor, information in the updated user profile to information in a medication-interaction database, and based on the comparing, determining whether there exists at least one interaction between the information in the updated user profile and the information in the medication-interaction database.

### TECHNICAL FIELD

The present disclosure relates to real-time, patient specific, personal medication and healthcare management and more specifically to a personalized medication and management and alert system and method that provides real-time information to users regarding potential adverse interactions between the user's current medications and other user characteristics such as the user's genetic makeup, food intake, illnesses, allergies and lifestyle.

### BACKGROUND

Personalized healthcare management as it relates to medicine is a medical procedure that separates patients into different groups based on their predicted response in how they activate or metabolize medications and how those medications affect disease. Medical decisions, practices, interventions and/or products are then tailored to the individual patient based on their unique medical profile. This medical profile can include a patient's unique genetic profile, their lifestyle and environment.

However, many of the existing personalized medication and management systems fall short of expectations for other reasons. Many systems do not capture real-time and updated patient medical data and therefore base their medical recommendations on a patient's outdated medical information. This may lead to providing incorrect medical recommendations with regard to a patient's pharmaceutical selection. Further, many personalized medication and management systems do not take into account the patient's genetic profile, thus leading to less-than-optimal medical recommendations and advice. Finally, many of the existing systems do not provide alerts or provide convenient access via a patient's computer or mobile device that can be shared with a doctor or a pharmacist prior to their prescribing a medication, or prior to an over the counter purchase. Pharmaceutical recommendations and guidance are often desired quickly while, for example, the patient is in a pharmacy and faced with a multitude of possible pharmaceutical selections. Up-to-date, correct, and easy-to-access information are essential aspects of any personalized medication and management system. Furthermore, government statistics show that over 35% of medications taken do nothing to help the patient and could even cause additional healthcare issues and possibly death.

### SUMMARY

In accordance with one aspect of the present disclosure, a personalized medication management and alert method is provided. In one embodiment, the method includes creating a user profile based at least upon received user genetic information, storing the user profile in memory on a computer, updating the user profile based on received user personal information, the received personal information including any medications currently used by the user, comparing, by a processor, information in the updated user profile to information in a medication-interaction database, and based on the comparing, determining whether there exists at least one interaction between the information in the updated user profile and the information in the medication-interaction database.

According to another aspect, a personalized medication management and alert system is provide. They system includes means for obtaining a sample from a user, the sample containing user genetic information and a data management system. The data management system is configured to create a user profile based at least upon the user genetic information contained in the user sample, store the user profile in memory on a computer, update the user profile based on received user personal information, the received personal information including any medications currently used by the user, compare information in the updated user profile to information in a medication-interaction database, and based on the comparing, determine whether there exists at least one interaction between the information in the updated user profile and the information in the medication-interaction database.

According to another aspect, a non-transitory computer-readable storage medium is provided, having stored therein instructions which, when executed by a processor, cause the processor to perform operations including creating a user profile based at least upon received user genetic information, storing the user profile in memory on a computer, updating the user profile based on received user personal information, the received personal information including any medications currently used by the user, comparing information in the updated user profile to information in a medication-interaction database, and based on the comparing, determining whether there exists at least one interaction between the information in the updated user profile and the information in the medication-interaction database.

According to another aspect, a personalized medication management and alert system is provided. The system includes means for obtaining a sample from a user, the sample containing user genetic information, the user genetic information containing single nucleotide polymorphisms (SNPs) for the user, and a data management system. The data management system is configured to create a user profile based at least upon the user genetic information contained in the user sample, store the user profile in memory on a computer, update the user profile based on received user personal information, the received personal information including any medications currently used by the user and at least one of user illnesses, user allergies, user lifestyle and user diet, compare, by a processor, information in the updated user profile to information in a medication-interaction database, based on the comparing, determine whether there exists at least one interaction between the information in the updated user profile and the information in the medication-interaction database, the at least one interaction including a medication-to-gene interaction, a medication-to-medication interaction, a medication-to-illness interaction, a medication-to-allergy interaction, a medication-to-lifestyle interaction, and a medication-to-diet interaction, and present on a display of a computer screen, information about the at least one interaction.

### BRIEF DESCRIPTION OF DRAWINGS

FIG. 1A a flow diagram showing the steps taken by the personalized medication management and alert system in accordance with an embodiment of the present disclosure;

FIG. 1B is a continuation of the flow diagram of FIG. 1A;

FIG. 2A is a flow diagram of the process flow of the personalized medication management and alert system in accordance with an embodiment of the present disclosure;

FIG. 2B is a continuation of the flow diagram of FIG. 2A;

FIG. 2C is a continuation of the flow diagram of FIG. 2B;

FIG. 3 represents exemplary screen shots presented on a user's computer or mobile device upon selection of Test Results Code from the My Profile tab in accordance with an embodiment of the present disclosure;

FIG. 4 represents exemplary screen shots presented on a user's computer or mobile device upon selection of My Medication from the My Profile tab in accordance with an embodiment of the present disclosure;

FIG. 5 represents exemplary screen shots presented on a user's computer or mobile device upon selection of Medical History from the My Profile tab in accordance with an embodiment of the present disclosure;

FIG. 6 represents exemplary screen shots presented on a user's computer or mobile device upon selection of Allergies from the My Profile tab in accordance with an embodiment of the present disclosure;

FIG. 7 represents exemplary screen shots presented on a user's computer or mobile device upon selection of My Lifestyle from the My Profile tab in accordance with an embodiment of the present disclosure;

FIG. 8 represents exemplary screen shots presented on a user's computer or mobile device upon selection of Contact Information from the My Profile tab in accordance with an embodiment of the present disclosure;

FIG. 9 represents exemplary screen shots presented on a user's computer or mobile device upon selection of Check Medication from the Check Meds tab in accordance with an embodiment of the present disclosure;

FIG. 10 represents exemplary screen shots presented on a user's computer or mobile device upon selection of My Warnings from the Check Meds tab in accordance with an embodiment of the present disclosure;

FIG. 11 represents exemplary screen shots presented on a user's computer or mobile device upon selection of Change Users from the Check Meds tab in accordance with an embodiment of the present disclosure;

FIG. 12 represents exemplary screen shots presented on a user's computer or mobile device upon selection of Family Sharing from the Check Meds tab in accordance with an embodiment of the present disclosure; and

FIG. 13 represents exemplary screen shots presented on a user's computer or mobile device upon selection of the Send Report tab in accordance with an embodiment of the present disclosure.

### DETAILED DESCRIPTION OF EXEMPLARY EMBODIMENTS

In an exemplary embodiment, the present disclosure provides a personalized medication management and alert system that provides real time data about potential adverse interactions between prescription and over-the-counter pharmaceuticals that the patient is using while considering other patient characteristics such as the patient's genetic makeup, their lifestyle, their current illnesses and allergies and their food intake, thus reducing the likelihood of negative side effects and overdoses. The personalized medication management and alert system and method of the present disclosure combines a user's personal medical and proprietary genetic information with current scientific and medical information, including real-time, newly discovered medical and medication information received from one or more databases, to create a user profile.

A user enters into the system by utilizing a software application on the user's computing device. The user can enter a medication name, an NDC number, and/or scan a medication barcode for a specific desired pharmaceutical that the user has been prescribed or is interested in purchasing. The system checks the user's profile to determine if the pharmaceutical may cause an adverse reaction or genetic threat to the user based upon the user's current use of other medications, food intake, illnesses, allergies, lifestyle and/or genetic profile. If a conflict does exist, the system generates a real-time alert to the user warning them of the conflict and/or provide a description of the adverse reactions. The system continues to monitor the user's personal healthcare profile and notifies the user as to any future changes in medications or healthcare innovations.

In one embodiment, the user may enter the name of the pharmaceutical and receive alerts via an application on the user's computer and/or mobile device. In addition to the alert, the user may receive or access reports that provide specific reasons why certain medications could be harmful, and, in some embodiments, provides alternate pharmaceutical recommendations to the user tailored to the user's specific healthcare and genetic profile.

In one embodiment of the present disclosure, a user provides their genetic information by means of a pharmacognetic (PGX) test. For example, the user receives a PGX test kit, which could, for example, be an FDA approved PGX kit, which contains a swab the user can use to swab the inside of their cheek to obtain a saliva specimen. The kit, containing the swab, is returned to a main facility, where the kit is matched to the specific user. The swab is analyzed and tests performed in order to obtain genetic information about the user. A report is generated and a user genetic profile is created. The report can, in one embodiment, identify over 20,000 genes and 700,000 gene variants as well as identify thousands of potential genetic anomalies that could lead to or could be tied to specific diseases.

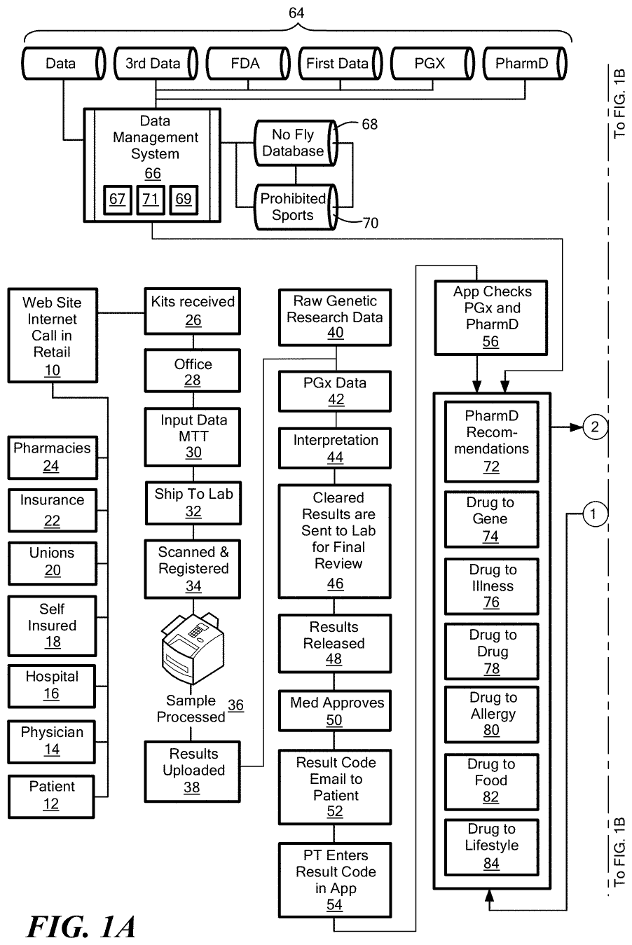

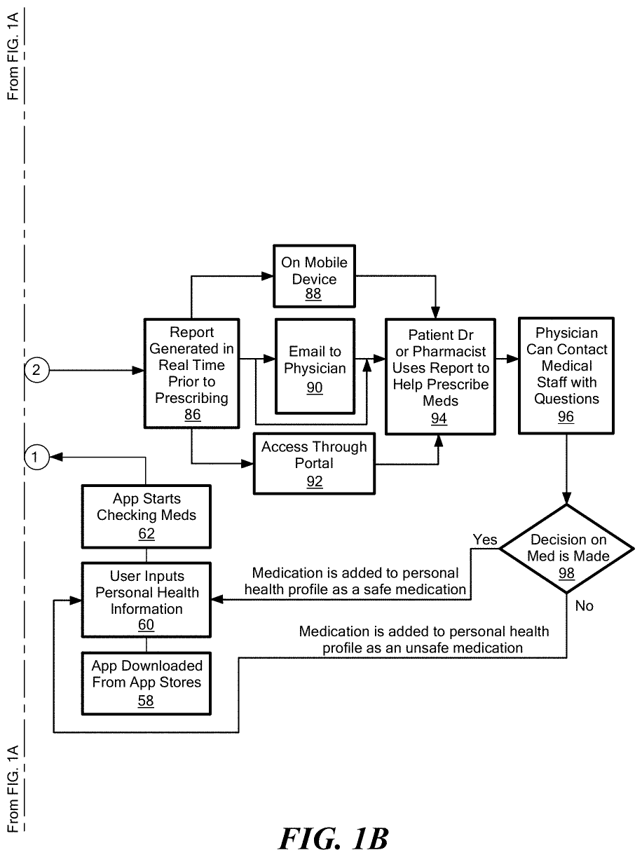

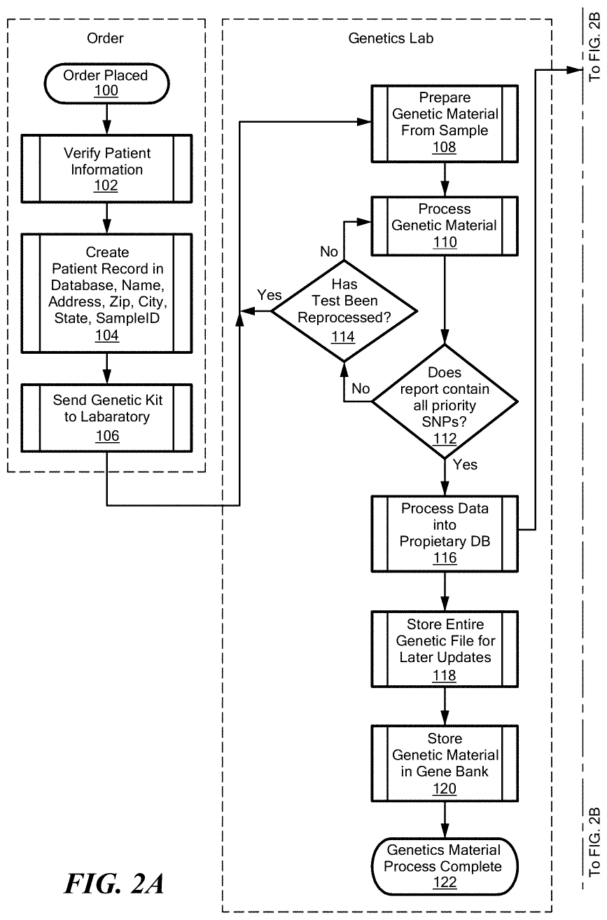

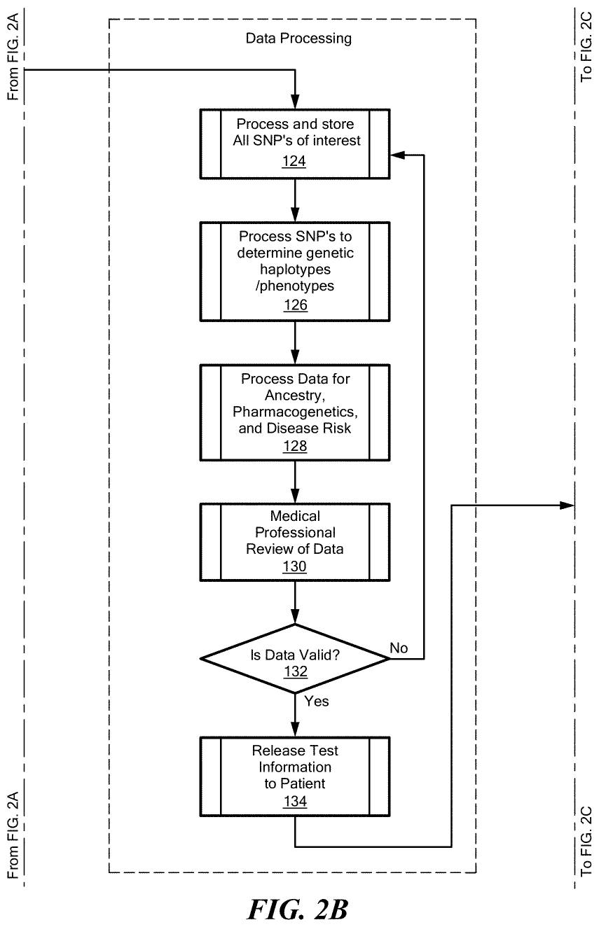

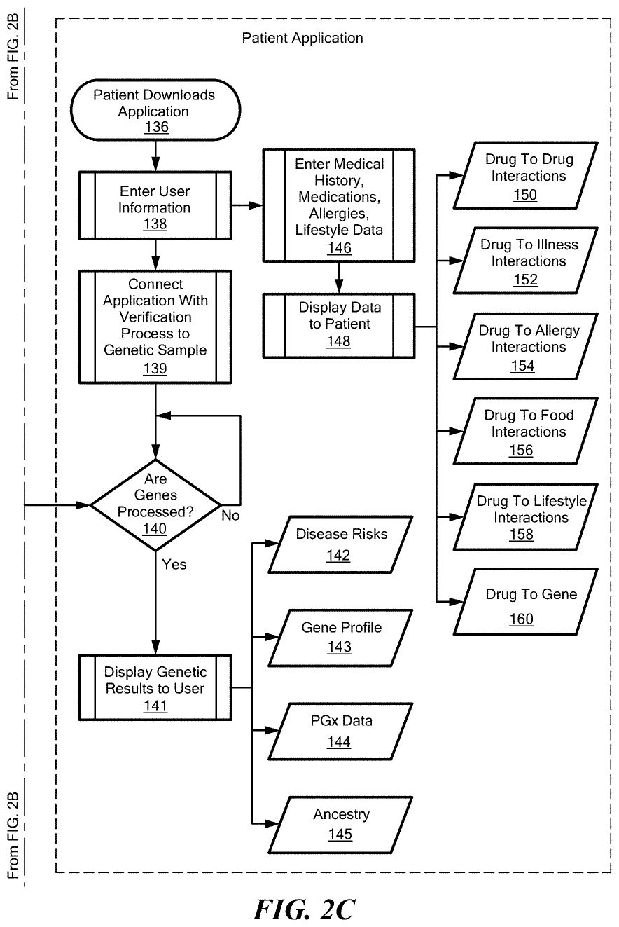

FIGS. 1A and 2A illustrate, in an exemplary embodiment, the personalized medication management and alert system in accordance with an embodiment of the present disclosure. Turning to FIG. 1A, at step 10, a person obtains a text kit by one of various different means. For example, the person may access the web site of the provider offering the personalized medication management and alert system of the present disclosure (“provider”). Alternately, the person can, via the Internet, visit other retailer websites to purchase the PGX test kit, the person may email or call into the provider to request a test kit, or the person may purchase the test kit from a retail store. The term “patient” shall be used herein to identify the person that submitted their saliva sample. It should be noted that the use of a PGX test kit is one process used to obtain the person's DNA and the person's DNA may be obtained via other means than a PGX test. Thus, the present disclosure is not limited in this regard.

While the term “patient” shall refer to the individual submitting their saliva sample for genetic testing, the term “user” may be a user of the platform such as an individual, i.e., a patient 12, or the user may be a physician 14, or a hospital 16 that has purchased the kit on behalf of patients, a self-insured company 18, or union 20 (i.e., companies responsible for managing and/or paying for healthcare expenses for individuals, a specific group, or multiple groups), an insurance company 22, or a pharmacy 24. The listing of exemplary “users” in FIG. 1A is not exhaustive and the present disclosure is limited to only those users listed. Anyone may contact the platform to order a PGX test. The PGX test kit may contain, in a non-limiting example, an FDA cleared collection device, i.e., a swab for allowing the patient to submit a saliva sample, a biohazard back to secure the sample, instructions and documents indicating how the user may register with the platform, and a consent document.

The platform receives the test kits with their specimens at step 26. The kits may have already been registered by the patient upon purchase or registered by the platform upon receipt of the kit. In one embodiment, the platform, upon receiving the kit, prints a kit manifest list detailing all barcodes on the kits and all written patient registration information and this information is uploaded into the platform database. The list may then be shredded to maintain proper HIPAA procedure. The kit received by a particular patient and registered into the platform is accessioned and approved by qualified medical personnel. A test is then ordered by a qualified physician resulting in a medically-ordered test. The kit is at this time referred to as an “activated” kit. In a non-limiting embodiment, the activated kit is received at a receiving office 28, the patient's data associated with the kit is input into a computer database at step 30, the activated kit is then shipped to a laboratory or testing facility at step 32, scanned and registered with the particular patient at step 34 and the contents of the patient's saliva sample processed at step 36. A kit that has been activated, approved and sent to a lab or testing facility is known as an “approved” kit. Again, the steps of receiving and processing the activated kits and testing the saliva sample provided in the kit is exemplary only and non-limiting. For example, the testing facility may be at the same location as the receiving office.

Once the sample from the received test kit has been processed, genetic information is obtained and uploaded at step 38. The platform, using raw genetic research data 40, extracts the PGX data from the uploaded processed information at step 42, interprets the PGX data at step 44, and the results are analyzed by a laboratory at step 46. The results of the analysis are released at step 48, the results reviewed and approved by a physician, lab worker, or other competent medical authority at step 50, to assure that there are no critical issues. If a critical issue exists, a nurse or physician may contact the patient to discuss the issue. It is noted that the analysis results may be reviewed by any physician, lab worker or other competent medical authority either associated with the platform or not associated with the platform.

A result code summarizing the genetic test results for the patient that submitted the sample is then sent to the patient at step 54. This could be, for example, a text or an email to the patient's computer or mobile device. As will be described in greater detail below, the patient receiving the result code may enter the code into a software application that the patient has obtained on their computer or mobile device which gives them access to the personalized medication management and alert system of the present disclosure. The application then checks the PGX information for the patient at step 56. By entering the result code in the application, the patient's genetic information may be linked to their personal health profile, which they have also entered into the application. The patient's genetic information and personal health profile are used by the platform to determine if any conflicts or interactions exist with regard to medications the patient is currently taking and a variety of other individual patient criteria.

Turning now to FIG. 1B, as mentioned above, the patient may download, at step 58, a software application on their computing or mobile device, which gives them access to the platform. The patient, via the application, is prompted to enter personal health information, at step 60. The personal health information may include such personal data such as the age and gender of the patient, where the patient lives, their marital status, their occupation, their height and weight, etc. The personal health information may also include current illnesses, any food or drug allergies that the patient suffers from, the amount of daily/weekly/monthly exercise the patient gets, whether the patient is a smoker, the types of food the patient eats, etc. The aforementioned list is not exhaustive and may be expanded to include other types of personal and personal health information about the patient.

The application also asks the patient to identify any medications the patient may be using. This may include prescribed pharmaceuticals, over the counter drugs, vitamins, supplements, etc. Once the patient has entered their personal health information and the current medications they are taking into their downloaded application, the platform then begins analyzing the medications, at step 62, and all of the entered information to determine if there are any interactions or conflicts between the current medications the patient is taking and any other criteria that the patient has identified such as their current illnesses, allergies, food and beverage intake and their current lifestyle (i.e., amount of exercise, daily activity, weight, etc.). Based on the entered information about the current medications the patient is taking, the platform also determines whether there are any conflicts between two or more medications. Thus, once the patient has downloaded the application onto their computing device and entered the requisite information as described above into the application, they will be presented with real-time, ongoing notifications regarding any interactions or conflicts based on the information about their health, lifestyle, and current medications that they submitted.

Returning to FIG. 1A, a series of third party databases 64 can be seen. Also shown is data management system 66, which is the entity that performs the algorithmic decisions by the platform in order to determine if any interactions exist for a particular patient. Third party databases 64 contain real-time information from complaint national medical reference libraries, databases, proprietary genetic libraries, and applications about medications, drugs, vitamins, and supplements, their ability to interact with other drugs, foods, allergies, etc. This information is can be accessed by data management system 66 on a real-time basis. Thus, as new drugs are entered into these databases or when new studies are performed about certain drugs and their interactions and side-effects, or any additional and/or new healthcare information is discovered and/or validated, this information can be accessed and used by the platform.

In addition to third party databases 64, additional proprietary databases can be created for specific use-cases. These proprietary databases gather their information from third party databases 64 and apply them to specific use cases. For example, a “no-fly database” 68 can be created to identify certain medications, vitamins, or supplements that pilots and aircraft operators should not be allowed to take. Similarly, a “prohibited sports” database 70 can be created to identify certain medications, vitamins, or supplements that athletes should not take. Databases 68 and 70 are exemplary only and any use-based proprietary databases can be created and its contents accessed on a real-time basis by data management system 66.

Data management system 66 receives real-time information from databases 64 and also receives the patient's genetic information that was obtained from the PGX test kit sample. Further, data management system 66 receives the patient's personal health information received at step 60. Based on this received information, data management system is able to determine whether there are any interactions or conflicts that the patient should be concerned with. These conflicts could be conflicts between two or more drugs, a drug and a patient's illness, a drug and an allergy, a drug and food and/or beverage being taken by the patient, between a drug and some aspect of the patient's lifestyle, and a drug and the genetic makeup of the patient.

In one embodiment, data management system 66 includes a traditional processor 67, a memory 69, and processing circuitry 71, which may include integrated circuitry for processing and/or control, e.g., one or more processors and/or processor cores and/or FPGAs (Field Programmable Gate Array) and/or ASICs (Application Specific Integrated Circuitry). The processing circuitry 71 may include and/or be connected to and/or be configured for accessing (e.g., writing to and/or reading from) the memory 69, which may comprise any kind of volatile and/or non-volatile memory, e.g., cache and/or buffer memory and/or RAM (Random Access Memory) and/or ROM (Read-Only Memory) and/or optical memory and/or EPROM (Erasable Programmable Read-Only Memory). Such memory 69 may be configured to store code executable by control circuitry and/or other data, e.g., data pertaining to communication, e.g., configuration and/or address data of nodes, etc. The processing circuitry 71 may be configured to control any of the methods described herein and/or to cause such methods to be performed, e.g., by the processor 67. Corresponding instructions may be stored in the memory 69, which may be readable and/or readably connected to the processing circuitry 71. In other words, the processing circuitry 71 may include a controller, which may comprise a microprocessor and/or microcontroller and/or FPGA (Field-Programmable Gate Array) device and/or ASIC (Application Specific Integrated Circuit) device. It may be considered that the processing circuitry 71 includes or may be connected or connectable to memory 69, which may be configured to be accessible for reading and/or writing by the controller and/or processing circuitry 71.

Based upon real-time information accessed from third party databases 64, PGX information obtained from the analyzed patient sample obtain from the PGX test kit, and personal health information received from the patient via the software application on their computing device, which includes identification of current medications, vitamins, and supplements being taken by the patient, processor 67 of data management system 66 determines whether any of the interactions shown in FIG. 1A exist and can make recommendations in this regard, at step 72.

For example, processor 67 of data management system 66 can determine if there are any drugs that the patient is currently taking that were could an adverse reaction to the patient based on the patient's genetic makeup as determined by the PGX data received from the patient's sample, at step 74. As an example, it may be determined that the patient carries the gene Cytochrome P450 2C19 (“CYP2C19”). Based on information accessed from third party databases 64 and the patient's personal health information, data management system 66 may determine that a patient with the CYP2C109 gene is very sensitive to the drug Doxepin, which the patient is currently prescribed. Thus, the patient may be notified on their computing device that there is a conflict between Doxepin and the gene CYP2C19. The notification may be, for example, “SEVERE CONFLICT!, Doxepin Conflict with CYP2C19. Consider an alternative drug, or consider prescribing doxepin at standard does and monitor the plasma concentrations of Doxepin and desmethyldoxepin to guide does adjustments.” In another embodiment, the patient may receive a notification that there is no conflict between any of the drugs the patient is currently taking and their genetic makeup.

Data management system 66 may then determine if there are any interactions between the medications the patient is currently taking and any illnesses the patient's is currently suffering from, at step 76. For example, if patient has indicated at step 60 that they are currently suffering from depression, data management system 66 may determine that use of Xanax® while suffering from depression is potentially harmful. If it is determined that such a conflict exists, a notification to this effect is generated and displayed to the patient. In another embodiment, the patient may receive a notification that there is no conflict between any of the drugs the patient is currently taking and any current illnesses they entered in step 60 as part of their personal health information.

Data management system 66 may then determine if there are any interactions between a medication that the patient is currently taking and any other medications the patient is taking, at step 78. For example, if the user indicates that they are currently taking Advil® and Warfarin®, which is a blood thinner, data management system 66 may determine that concurrent use of the two medications may increase the risk of bleeding. If such a conflict exists, a notification is generated and displayed to the patient on their computer or mobile device. In another embodiment, the patient may receive a notification that there is no conflict between any of the drugs the patient is currently taking as entered in step 60 as part of their personal health information.

Data management system 66 may then determine if there are any interactions between a medication that the patient is currently taking and any allergies the patient suffers from, at step 80. For example, if the patient has indicated that they currently have an allergy to Amoxicillin®, data management system 66 may generate an adverse notification warning the patient that it might be harmful to use Amoxicillin®, any medications similar to Amoxicillin®, or other medications in combination with Amoxicillin®. If such a conflict exists, a notification is generated and displayed to the patient on their computer or mobile device. In another embodiment, the patient may receive a notification that there is no conflict between any of the medications the patient is currently taking and the allergies they entered in step 60 as part of their personal health information.

Data management system 66 may then determine if there are any interactions between a medication that the patient is currently taking and any food or beverages that the patient typically consumes, at step 82. For example, if the patient has indicated that they are currently prescribed Plavix® and data management system 66 may generate an adverse notification warning the patient warning them that it might be harmful to use Plavix® with citrus-based fruits. If such a conflict exists, a notification is generated and displayed to the patient on their computer or mobile device. In another embodiment, the patient may receive a notification that there is no conflict between any of the medications the patient is currently taking and their regular food intake as entered in step 60 as part of their personal health information.

Data management system 66 may then determine if there are any interactions between a medication that the patient is currently taking and the patient's current lifestyle, at step 84. The patient, as part of their personal health information entered on their downloaded software application, at step 60, may have indicated, for example, that as part of their lifestyle, they exercise 2 days a week, work at a sedentary job, get an average of 6-7 hours of sleep a night, smoke regularly, and consume 5-7 alcoholic beverages a week. Data management system 66 may then determine whether there are any current interactions between the medications the patient has indicated they are taking with any of the patient's current lifestyle parameters. For example, data management system 66 may determine that it may be harmful to take Xanax® with the amount of alcohol the patient takes on a weekly basis. If it is determined such an interaction exists, a notification to this effect is generated and displayed to the patient on their computing or mobile device. In another embodiment, the patient may receive a notification that there is no conflict between any of the medications the patient is currently taking with any of the lifestyle parameters as entered in step 60 as part of their personal health information.

In each case above, processor 67 of data management system 66 compares the real-time scientific and medical information obtained from third party databases 64 and/or other proprietary databases, e.g., 68 and 70, with the patient-specific genetic information obtained from the sample specimen provided by the patient via the PGX test kit and the personal health information the patient entered on their downloaded software application at step 60. Based upon these comparisons, data management system 66 is able to provide notifications to the computing device of the patient, informing them if there any adverse conflicts, as described above. Note that as current scientific and medical data changes or new data becomes available, data management system 66 is able to capture this information from third party databases 64 and provide real-time and accurate notifications to the patient.

The above-mentioned notifications can include a warning of a conflict, an indication as to the severity of a conflict (i.e., mild, moderate, severe) or an indication that there is no conflict. Thus, the patient receives a high level of confidence when it comes to determining if there are any potential medical hazards that the patient needs to be concerned with when the patient purchases a drug. Further, it should be noted that the order shown in FIG. 1A in which the interactions are determined is exemplary only. Thus, data management system 66 may determine if a conflict exists between any of the interactions (i.e., drug-gene, drug-illness, drug-drug, drug-allergy, drug-food, drug-lifestyle) in any order.

Returning to FIG. 1B, once recommendations have been made at step 72 and notifications sent to the patient on their computing or mobile device, a report is generated in real-time at step 86. The report may be forwarded to the patient, physician and/or pharmacist via a text on their mobile device at step 88, via an email, at step 90, or via access through the platform's portal, at step 92. At step 94, the report can be reviewed and used by the physician or pharmacist to prescribe the medication that the patient wants to purchase or use, provided the report has indicated no adverse effects. The prescribing physician or pharmacist can contact one of the platform's medical experts if they have questions about the report, at step 96. If the physician or pharmacist determines that the medication that the patient wishes to use is clear of any conflicts, the medication is added to the personal health profile of the patient as a “safe medication.” If the physician or pharmacist determines that the medication that the patient wishes to use is causes a conflict, the medication is added to the personal health profile of the patient as an “unsafe medication.”

Turning now to FIGS. 2A-2C, an exemplary embodiment of the personalized medication management and alert system of the present disclosure is shown. In particular, the flow diagram of FIG. 2A shows the process flow from the ordering of the PGX test kit to the processing of the patient's genetic sample material taken from the test kit. An order for the PGX test kit is placed by a user at step 100. As described above and shown in FIG. 1A, the order can come from a variety of “users” and may be placed in a variety of ways including the user placing the order directly from the platform's website, via a call by the user to a call center associated with the platform, via an email, or the user may purchase the PGX test kit from an online or brick-and-mortar retail store that sells the PGX test kits.

When the platform receives the order, they verify the patient's information at step 102 and create a patient record in a database that can include, for example, the patient's name and address, and an identification number that is assigned to the patient, at step 104. The patient then obtains a saliva sample by following instructions provided in the PGX test kit and submits the PGX kit with their genetic sample by mailing the kit containing the patient's sample to a predetermined location, i.e., an office or warehouse. The received PGX test kit is then sent to a genetics laboratory for interpretation, at step 106.

At the genetics laboratory, the patient's saliva sample is analyzed and genetic material is extracted from the sample at step 108 and processed at step 110. The processed genetic material is analyzed to determine if the material contains all priority single nucleotide polymorphisms (“SNPs”) and a report is prepared at step 112. In the context of the present disclosure, “priority” SNPs have been medically validated, and these priority SNPs are added to the user's personal healthcare profile. If the report does not contain all the priority SNPs for this patient, and if the test has not been reprocessed, at step 114, then the genetic material is processed again, at step 110. If the test has been reprocessed then the genetic material is again taken from the sample at step 108. If the report does contain the necessary priority SNPs, the genetic data for this particular patient is processed and saved in a proprietary database, at step 116, and the entire genetic profile file for the patient is saved, at step 118, and can be updated in the future if necessary. Finally, the genetic material taken from the patient's sample can be stored at step 120, thus completing the genetic material process portion of the present disclosure, at step 122.

From step 116 of FIG. 2A, which is the processing of the patient's genetic data into a proprietary database, the process continues in FIG. 2B, which depicts an exemplary embodiment of the data processing portion of the present disclosure. At this juncture in the process, the PGX test kit has been received by a kit receiving facility and the contents of the kit, i.e., the patient's saliva sample, has been tested for genetic material. All SNPs that have been clinically validated for this particular patient are processed and stored, at step 124. The selected SNPs that have been processed are then analyzed to determine genetic haplotypes and/or phenotypes, at step 126. The patient's genetic data is processed in order to obtain information about the patient's ancestry, pharmacogenetics and disease risks, at step 128. In one embodiment, the processes of steps 124, 126 and 128 are performed by processor 67 of data management system 66.

The processed patient genetic data is reviewed by a qualified medical professional, at step 130, who determines, at step 132 if the genetic data is valid. If the medical professional determines that the genetic data is not valid, the SNPs of interest are again processed at step 124 and steps 126-130 are repeated until it is determined, at step 132, that the processed data is valid. The test information containing the valid data is released to the patient, at step 134. In one embodiment, the patient receives an electronic communication, i.e., an email or text message with a personal code. The patient enters the personal code into the software application running the system on their computing device and their genetic information is automatically mapped to their personal healthcare profile.

At this point in the process, the patient has submitted their saliva specimen via the PGX test kit, and the specimen has been analyzed and genetic material extracted. The genetic material has been analyzed, the SNPs processed, and the resulting data has been sent to the patient. FIG. 2C depicts the flow process from the patient side, when the patient downloads a software application on their computing or mobile device in order to obtain the benefits of the personalized medication management and alert system of the present disclosure. Referring to FIG. 2C, the patient downloads the software application, at step 136, and, when prompted by the application, enters into their computing or mobile device, their personal and medical information via the application, at step 138. At step 139, the software application is connected with the genetic material verification process (step 132), and if all the patient's genetic information has been processed, at step 140, the genetic information results are displayed on the patient's computing or mobile device, at step 141. The genetic information may include, for example, patient-specific disease risks 142, a gene profile 143, PGX data 133 and ancestry 145.

As described above, the information entered into the software application by the patient at step 138 may include things like the patient's medical history, including all medications the patient is currently taking, any allergies the patient suffers from, lifestyle and personal information, such as, for example, age, occupation, amount of exercise, amount of alcohol of tobacco intake, etc., step 146. This information may be displayed to the patient, at step 148 and, in one embodiment, the patient may be asked to verify this information.

As described above, once the patient has entered their personal and medical information into the software application, data management system 66 performs a plurality of comparisons and determines whether any medications the patient is currently taking would adversely interact with other patient characteristics. For example, data management system 66 determines if any medications the patient is taking adversely interacts with: other medications taken by the patient, i.e., drug-to-drug interactions 150, patient illnesses, i.e., drug-to-illness interactions 152, patient allergies, i.e., drug-to-allergy interactions 154, food or beverage taken by the patient, i.e., drug-to-food interactions 156, the patient's lifestyle, i.e., drug-to-lifestyle interactions 158 and the patient's genetic makeup, i.e., drug-to-gene interactions 160.

Thus, when a patient wishes to purchase new medication, or a doctor wishes to prescribe a patient a new medication, the patient enters the name of the medication in the software application which embodies the personalized medication and management system of the present disclosure, data management system 66 determines if there are any interactions at steps 150-160 and can display the result of these determinations to the patient on their computing/mobile device. Because data management system 66 obtains real-time data from third party databases 64, data management system 66, via the software application installed on the patient's computing/mobile device is able to provide the patient with up-to-date information regarding interactions that might adversely affect the patient. This information can be crucial to a patient who is purchasing medication for the first time at, for example, a pharmacy, and wants to know if they will suffer any adverse effects. Further, a pharmacist or doctor would also want to know if the medicine they are about to prescribe or dispense would have an adverse reaction to the patient based on the patient's medical and personal history. The system can display or provide an audible communication to the patient that informs the user of any and all side effects associated with a particular medication. For example, when the patient scans the bar code of a medication, or enters the name of the medication into the application, the patient is presented with potential side effects; this information can be invaluable to a patient, and is a substitute for the patient having to read tiny fine print on the medication bottle. Further, the patient is being presented with current, real-time information as opposed to side effects information which may be outdated, and thus obsolete.

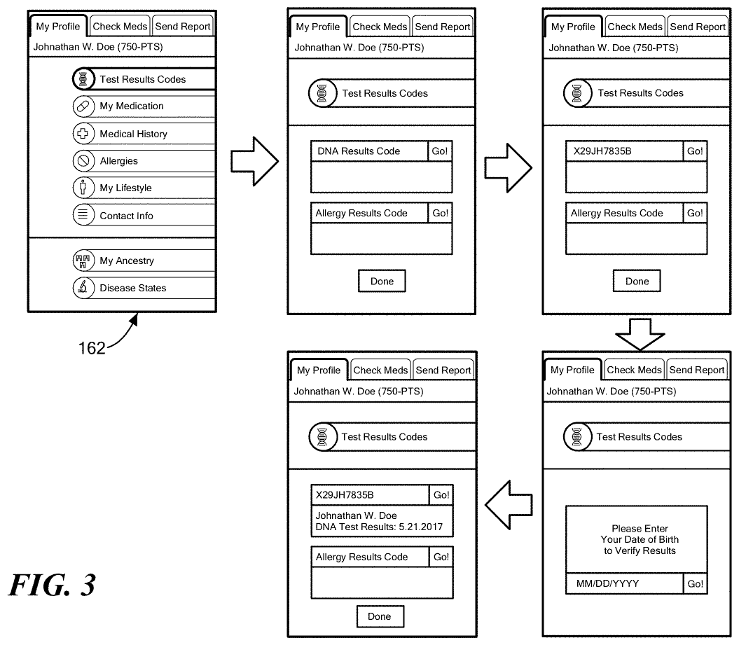

FIGS. 3-13 illustrate exemplary screen shots of a patient's mobile device employing the software application that encompasses the personalized medication management and alert system of the present disclosure. Depending upon the selections by the user, different screens of information are displayed to the patient. The screen shots in FIGS. 3-13 are exemplary only and different screen layouts, selection tabs, and message prompts may be used. Further, not all scenarios are depicted in FIGS. 3-13 and only exemplary scenarios are shown. Thus, the screen layouts, message prompts and selection tabs are not meant to limit the present disclosure in any way. FIGS. 3-13 illustrate how a patient can easily enter the information needed to allow the platform to provide to the patient real-time interaction information as shown in the flowcharts of FIGS. 1A through 2C and discussed above.

For example, in FIG. 3, a screen 162 is shown to a patient who has registered form the platform and has downloaded the software application encompassing the personalized medication management and alert system of the present disclosure. It is assumed that the patient has already registered into the system and submitted a saliva sample via the PGX test kit, and that this sample has been analyzed and the result of the genetic sample analysis emailed to the patient in the form of a DNA results code. On their computing/mobile device, under the My Profile tab, the user selects Test Results Code, and enters their DNA results code that was previously sent to them. Once this information has been entered into the application, the patient may be prompted to enter their date of birth in order to verify the identity of the patient.

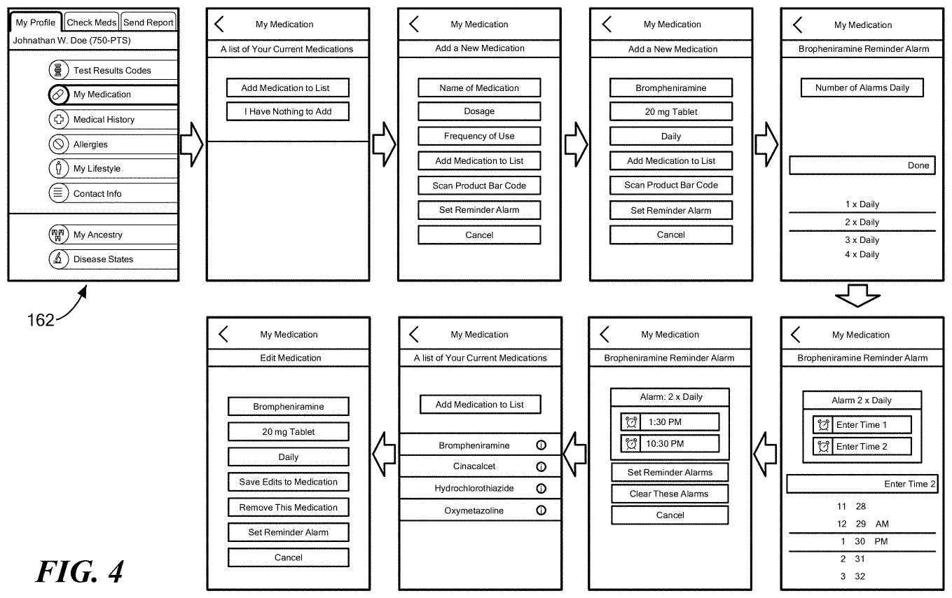

In FIG. 4, on their mobile device, the patient selects My Medication and is prompted to add the name of any medication the patient is taking (if any) and that has not already been entered. For example, the patient selects Add Medication to List and is prompted to enter the name of the medication, the dosage, and the frequency of use. In one embodiment, the patient can scan the bar code of the medication instead of entering its name. In this example, the patient enters the name of the medication, Brompheniramine, its dosage, i.e., 20 mg, and its frequency of use, i.e., daily. The patient may also select Set Reminder Alarm which would result in an alarm (or multiple alarms, i.e., twice daily, three times daily, etc.) on the patient's mobile device, reminding the patient to take the next dose of the medication that was entered. The patient can set the alarm times, if any, and then add additional medications to the list, if any. This process continues until the patient no longer has any new medications to add.

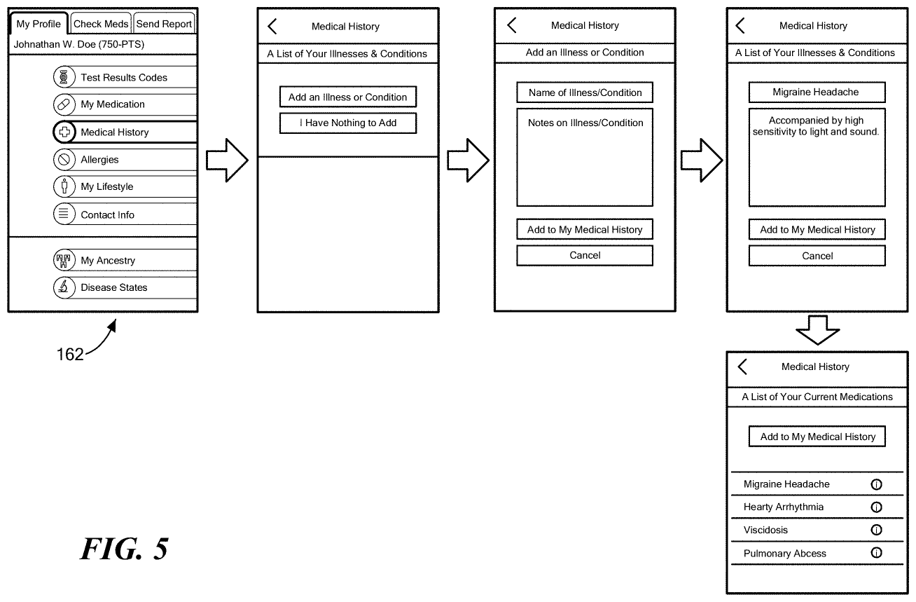

Referring to FIG. 5, still under the My Profile tab, the patient may then enter their medical history. In this example, the patient enters the name of the illness, i.e., migraine headache, and any accompanying notes, i.e., high sensitivity to light and sound. Additional illnesses and accompanying notes, if any can then be entered in a similar fashion.

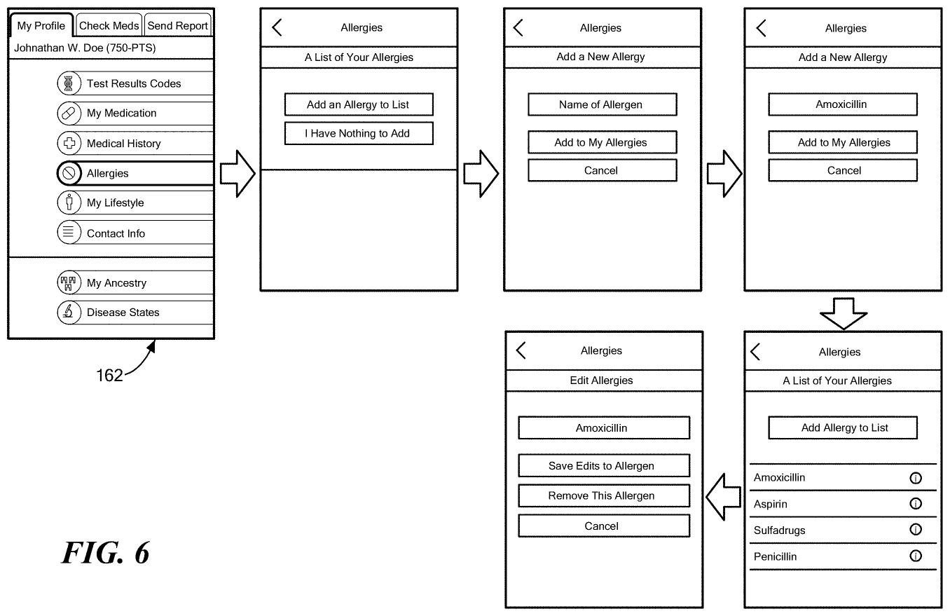

FIG. 6 depicts exemplary screens still under the My Profile tab that are shown to the patient when the patient enters allergies they currently suffer from under the My Profile tab. As shown in FIG. 6, the patient has entered that they are allergic to Amoxicillin, Aspirin, Sulfadrugs and Penicillin.

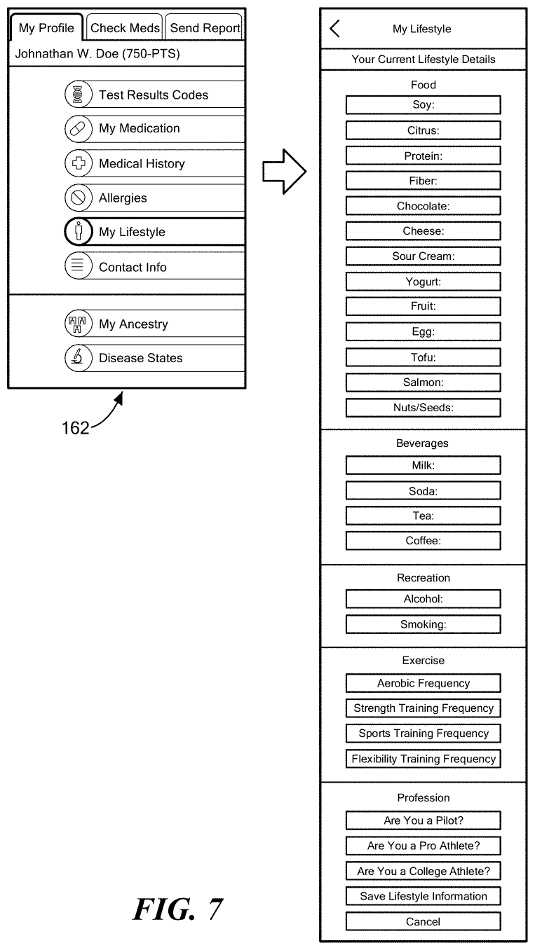

FIG. 7 shows exemplary screens, still under the My Profile tab, that follow the selection of the My Lifestyle tab by the patient. The patient is then prompted to enter details about their current lifestyle. In this embodiment, the patient has entered information about their food and beverage intake, their recreational activities, exercise and their profession.

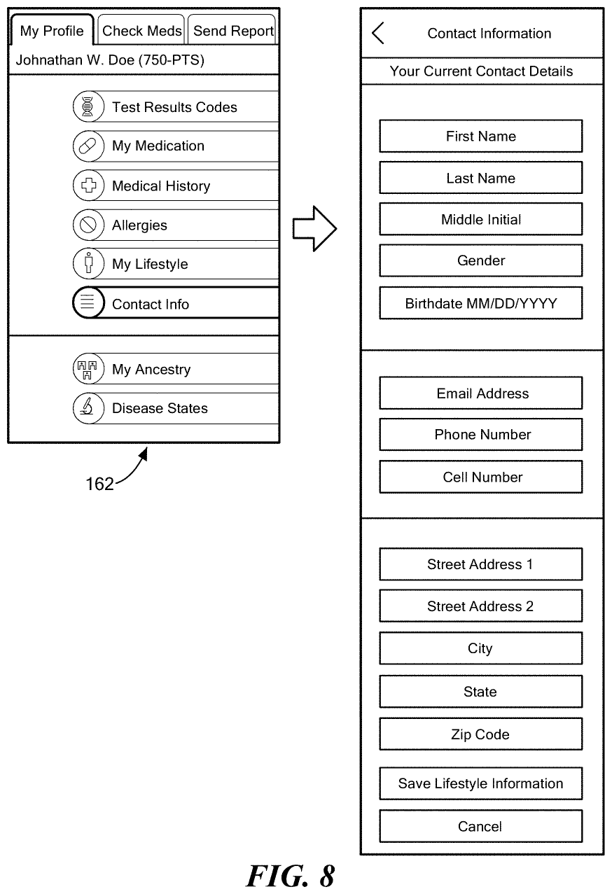

FIG. 8 represents exemplary mobile device screens, still under the My Profile tab, when a patient enters their contact information into the software application.

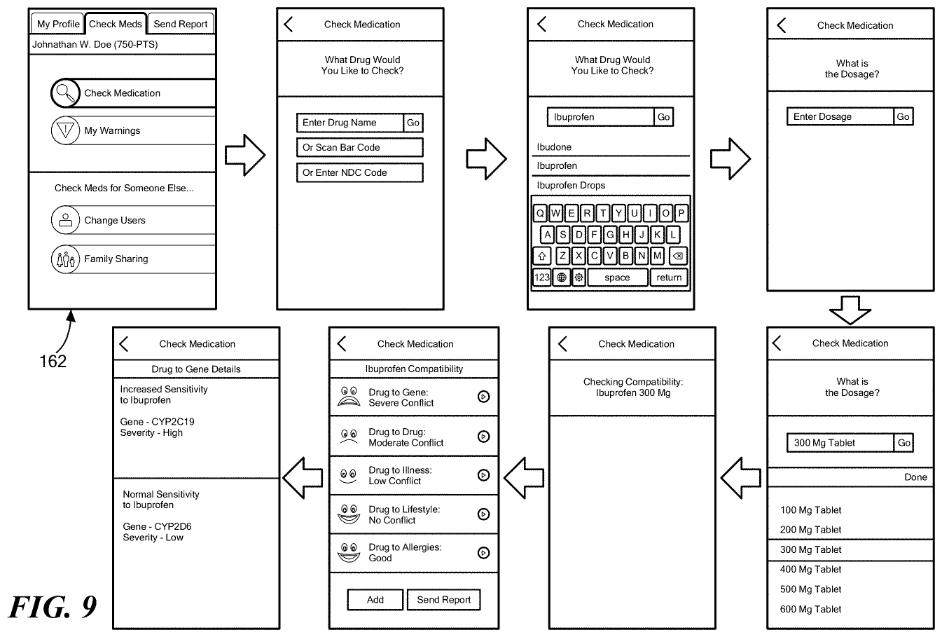

FIG. 9 illustrates a sequence of mobile device screens that a patient may see when the patient, under the Check Meds tab, wants to determine if a medication that they are taking or are about to purchase may have harmful effects in view of the patient's personal characteristics that they have already entered into the software application that encompasses the personalized medication and management and alert system of the present disclosure. In the exemplary embodiment of FIG. 9, the patient is prompted to either enter the name of the medication, or enter its National Drug Code (NDC) code or scan its bar code. In this example, the patient enters the name of the medication, Ibuprofen, and its dosage. Data management system 66 then determines if there are any interactions between Ibuprofen at 300 mg, and any of the patient's characteristics that have been previously entered into the software application.

In this example, when the patient selects the Drug to Gene icon, a Drug to Gene Details screen appears. This screen shows that there is a severe conflict between Ibuprofen and the patient's genetic makeup, specifically the CYP2C19 gene. There is also a low severity conflict with respect to Ibuprofen and the CYP2D6 gene. Under the Check Medication screen, it can be seen that there is also a moderate conflict between Ibuprofen and other drugs the patient is taking, and a low conflict between Ibuprofen and the patient's current illnesses. In one embodiment, the system may also generate a real-time report identifying similar medications, if there are any, that might have a potential issue. The report may also list alternate medications that would have no potential conflicts and/or similar medications that have no genetic research associated with them so that a physician is presented with all the available and necessary information regarding that particular patient and their medication so that the physician can provide the best medical treatment decision for that patient.

In the current example, there is no conflict between Ibuprofen and the patient's lifestyle and there is no conflict between Ibuprofen and any of the patient's allergies. When the patient selects the Drug to Drug button/icon, or the Drug to Illness button/icon, they will be presented with screens similar to the one shown in FIG. 9 for Drug to Gene Details, except that specific information related to conflicts between patient medications or conflicts between patient medication and patient illnesses will be shown.

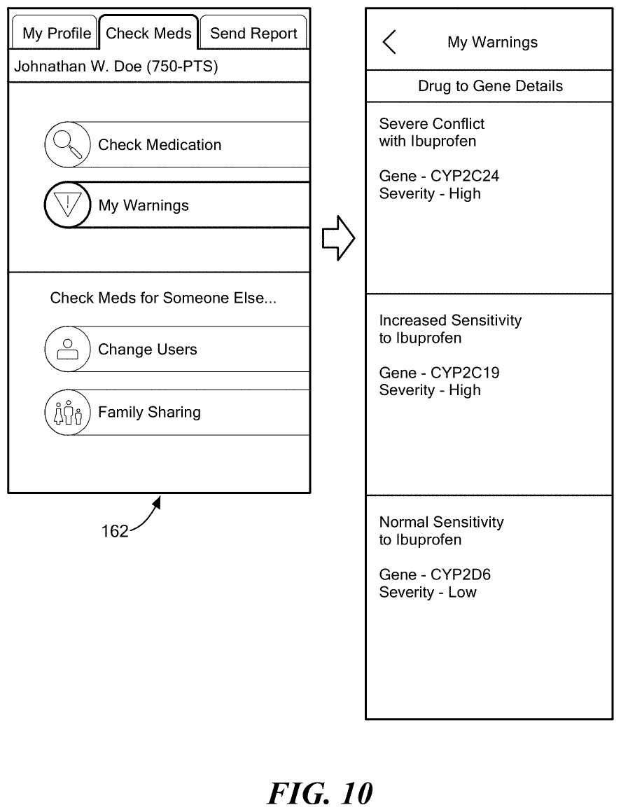

Referring now to FIG. 10, under the Check Meds tab, the patient may select the My Warnings button/icon. This selection will present a screen or series of screens that allows the patient to view all conflicts. Although only the Drug to Gene conflicts screen is shown in FIG. 10, if the patient had other conflicts, for example, between medications and lifestyle, or between medications and allergies, these conflicts can also be presented to the patient on corresponding screens. Thus, selecting the My Warnings button/icon will allow the patient to conveniently view all conflicts that patient may have, whether the severity is low, medium, or high and regardless of the type of conflict, i.e., drug-to-drug, drug-to-gene, drug-to-food, etc.

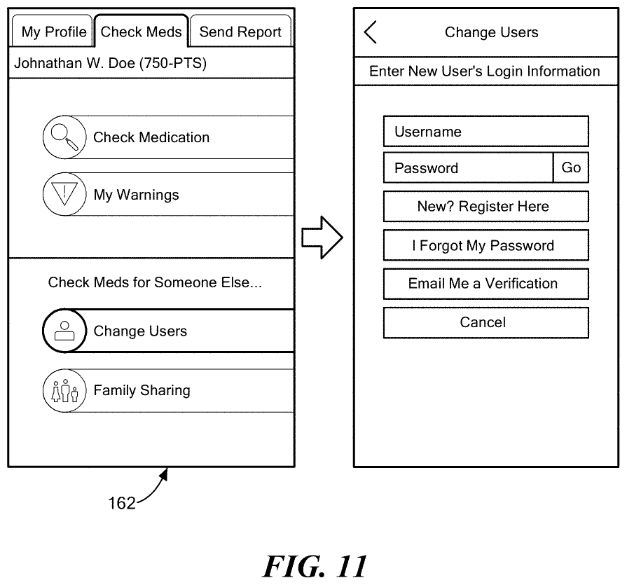

Using the software application, the patient may be presented with the option of checking medication allowances or conflicts for someone else, i.e., another registered user of the platform. A shown in FIG. 11, the Change Users button/icon may be selected. Doing this results in the presentation of a New User's login screen, where a different user may enter their identification information. Screens will be presented in the same fashion as described above, except the information will relate to the new user and show the new user's conflicts, if any.

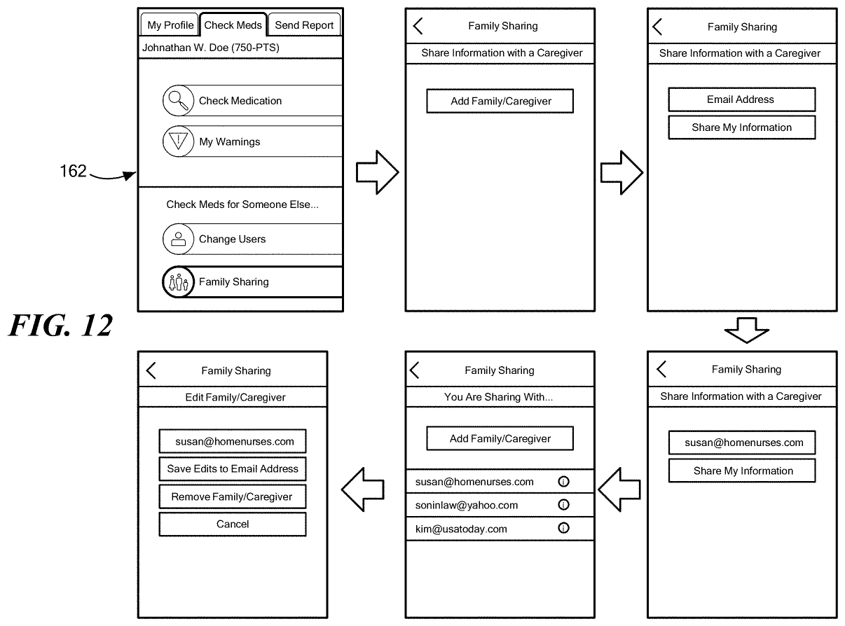

FIG. 12 depicts the Family Sharing option under the Check Meds tab. In this embodiment, by selecting Family Sharing, the patient is presented with the option of sharing their medication conflict information with others, i.e., other members of their family, or friends. This option may be valuable if, for example, the patient's spouse is shopping at a pharmacy and sees a sale on medication and wants to check to see if that medication would conflict with any of their spouse's characteristics. Another advantage is the option for the patient to share their medication conflict information with another caregiver. For example, if the patient is changing doctors, they can simply add the contact information of the new doctor and that doctor now has access to the patient's medication conflict information. This is an invaluable tool to the new doctor as it allows the new doctor to get a “snapshot” of the patient's medical history and will facilitate treatment and the prescribing of medication to the patient.

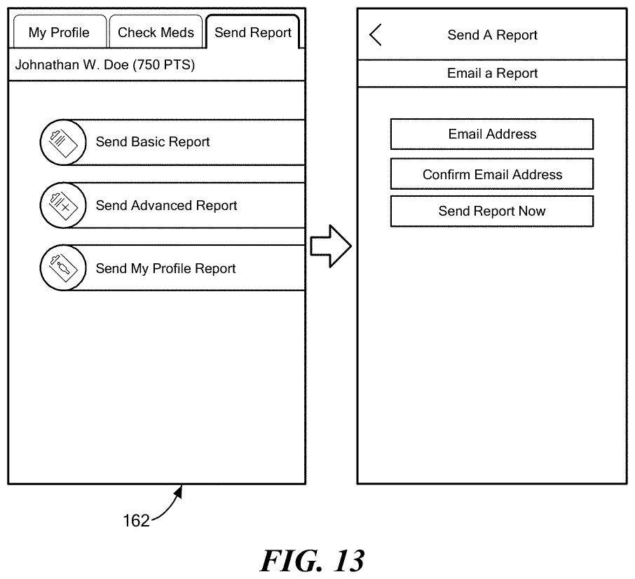

FIG. 13 shows screens after the patient has selected the Send Report tab. In this embodiment, the patient can elect to have a report sent to a selected recipient. The report can include the patient's personal and medical information, and information related to their genetic characteristics as a result of the PGX test kit analysis. The system generates multiple report options that include a basic patient report which identifies all medications that patient is taking, any existing illnesses, allergies, or lifestyle issues so that a physician is provided with the most up to date patient information prior to seeing the patient. Another report can be generated that identifies information on specific drugs that are chosen and makes recommendations. An advanced report can be generated that provides detailed, comprehensive information on all known drugs that could present medical issues when considering the user's personal medical profile. These reports provide the physician with a comprehensive tool that assists the physician in prescribing the best treatment for that particular patient.

The system and method of the present disclosure relates to a genetics-based system that allows users/patients to use a saliva swab in order to obtain genetic material and where the system can then build a genetic profile for the user from the obtained genetic sample and determine if there any interactions between the user's genetic makeup and any medications the user is currently taking or wishes to take.

In one embodiment, a user that has previously registered with the platform, obtains a test kit, and following directions, obtains a saliva sample. The sample is then returned to a processing center, i.e., data management system 66, where the sample is matched to the registered user. The genetic sample is then tested and analyzed. A genetic sequencing of the sample is obtained and stored in the user's genetic profile. The genetic profile may include information such as the SNPs for the user as well as their copy number variants (CNVs). The profile may include translation table for specific genes and associated SNPs with haplotype/genotype/phenotype, etc.

The user's data will then be processed to match the SNPs to an appropriate phenotype for particular genes. In a non-limiting example, if a user had an snp1 with AA and snp2 with CC and snp3 with TT, they would be designated an extensive metabolizer. This example holds true for all genes except for those which need a CNV to provide accurate details. The CNV is a nomenclature for those genes that a human may have multiple copies of and therefor has a multiplicative power. Therefore, if the phenotype for the above example is “extensive metabolizer” and the user has multiple copies, it may cause them to have an even higher rate of metabolization. In one embodiment, in the case of a gene having a CNV, there can be a database translation table for this, which can be consulted if needed.

In addition to determining gene-to-medication interactions, the system of the present disclosure can also determine one or more of the following additional interactions: medication-to-medication, medication-to-illness, medication-to-allergy, medication-to-food (and drink); and medication-to-lifestyle. Some or all of the above interactions may obtained in the following manner: In the application, for example, when the user downloads an application to their mobile device or computer, or when the user registers for the system in some other manner (visits website, email, etc.), the user is prompted to enter their food type consumption and frequency, for example, orange juice 2× per day, and also to enter any known allergies, current medications they are taking, any current or known health conditions, and answer questions about their current lifestyle (frequently exercise, smoke a pack a day, consume 5 alcoholic beverages a week, sedentary office job, etc.). Once this information is entered it is stored, for example, in a database table associated with that user. The system then obtains real-time data regarding potential interactions between medications that the user has indicated they are currently taking and various other user criteria. The system then determines whether there are any current known medication to gene, medication to medication, medication to allergy, medication to lifestyle and medication to food interactions.

The system can then format and display these interactions to the user. For example, the interactions are displayed on the user's cellular device via iOS, Android, or Web Applications. The display may include, for example, the interaction severity, the items that are interacting, and a message pertaining to the interaction. In addition to displaying this information to the user, the system may provide an interactive screen that enables the user to send the information about the interactions in a report, for example a pdf report, to anyone of their choosing. This could be a family member, pharmacy, physician, etc.

The system also has a portal-based patient registration section that is custom specific to physicians and hospitals, which allows the medical facility to register a new patient into the system. A specimen sample is taken from the patient so that a genetic test can be performed and a medical staff in the facility can check any medications for that patient prior to prescribing medication, authorizing surgery, or discharging the patient. For example, there are certain medications that do not work for all patients. One of these medications is OxyContin®, which is a commonly used powerful pain medication. If the patient cannot properly metabolize OxyContin®, they will receive no therapeutic result from this medication. The patient may only suffer from adverse side effects if this medication is prescribed, which could make the patient worse or even cause death.

A specific use case will now be discussed. In one embodiment, a user orders an FDA-cleared test kit online or is tested by their employer. At this point, the user is not ordering a test, but is just purchasing a collection kit. The kit, with the user's sample, is sent back to the platform and reviewed, approved, and accessioned by a medical department affiliated with the platform and a lab order (requisition) is created. The kit is sent to a lab as a physician-ordered test. The test is treated at the lab as a physician-ordered test and (Clinical Laboratory Improvement Amendments (CLIA) approved. The lab tests the sample and then a pathologist signs off on the test and sends the raw data back to the platform. The lab uses the platform's system to receive test orders and upload raw test results. The platform's medical department reviews each result looking for any potential conflicts and if there are conflicts, the patient is notified and the platform offers to resolve the issues because there are usually multiple doctors involved writing scripts that should not be taken together. This could be due, for example, because the user has dealt with several physicians, each of whom have prescribed different medications.

The platform identifies any potential conflicts between these medications, in the manner described throughout this disclosure. The platform releases the results to the user, and may generate reports, which can be sent to the user's physicians. In one embodiment, the user is now a customer of the platform, and the platform retains ownership of the patient data. The date is HIPAA protected and the user must approve release of the information to their physicians, completing the HIPAA cycle. Thus, in one embodiment, the platform is a licensed medical company prescribing testing and supporting the user who becomes a patient of the platform.

As will be appreciated by one of skill in the art, the concepts described herein may be embodied as a method, data processing system, and/or computer program product. Accordingly, the concepts described herein may take the form of an entirely hardware embodiment, an entirely software embodiment or an embodiment combining software and hardware aspects all generally referred to herein as a “circuit” or “module.” Furthermore, the disclosure may take the form of a computer program product on a tangible computer usable storage medium having computer program code embodied in the medium that can be executed by a computer. Any suitable tangible computer readable medium may be utilized including hard disks, CD-ROMs, electronic storage devices, optical storage devices, or magnetic storage devices.

Some embodiments are described herein with reference to flowchart illustrations and/or block diagrams of methods, systems and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer (to thereby create a special purpose computer), special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable memory or storage medium that can direct a computer or other programmable data processing apparatus to function in a particular manner, such that the instructions stored in the computer readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Computer program code for carrying out operations of the concepts described herein may be written in an object oriented programming language such as Java® or C++. However, the computer program code for carrying out operations of the disclosure may also be written in conventional procedural programming languages, such as the “C” programming language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

1. A personalized medication management and alert method, the method comprising:
creating a user profile based at least upon received user genetic information;
storing the user profile in memory on a computer;
updating the user profile based on received user personal information, the received user personal information including any medications currently used by the user;
storing information in a medication-interaction database, the information in the medical-interaction database obtained from at least one medical reference source;
updating the stored information in the medication-interaction database to include current scientific and medical data changes as they become available;
comparing, by a processor, information in the updated user profile to the updated information in the medication-interaction database; and
presenting on a user's computing device, a visual display indicating if there are any interactions between a medication to be taken by the user and the user's genetic makeup, other medications taken by the user, the user's current illnesses, the user's lifestyle, the user's diet, and the user's allergies.

2. The method of claim 1, wherein the user genetic information is obtained from a pharmacogenetic test.

3. (canceled)

4. The personalized medication management and alert method of claim 1, wherein the received genetic information contains single nucleotide polymorphisms (SNPs) for the user.

5. The personalized medication management and alert method of claim 1, wherein the user personal information includes at least one of user illnesses, user allergies, user lifestyle and user diet.

6. (canceled)

7. A personalized medication management and alert system, the system comprising:
means for obtaining a sample from a user, the sample containing user genetic information; and
a data management system configured to:
create a user profile based at least upon the user genetic information contained in the user sample;
store the user profile in memory on a computer;
update the user profile based on received user personal information, the received personal information including any medications currently used by the user;
store information in a medication-interaction database, the information in the medical-interaction database obtained from at least one medical reference source;
update the stored information in the medication-interaction database to include current scientific and medical data changes as they become available;
compare information in the updated user profile to the updated information in the medication-interaction database and
present on a user's computing device, a visual display indicating if there are any interactions between a medication to be taken by the user and the user's genetic makeup, other medications taken by the user, the user's current illnesses, the user's lifestyle, the user's diet, and the user's allergies.

8. The system of claim 7, wherein the user genetic information is obtained from a pharmacogenetic test.

9. (canceled)

10. The system of claim 7, wherein the received genetic information contains single nucleotide polymorphisms (SNPs) for the user.

11. The system of claim 1, wherein the user personal information includes at least one of user illnesses, user allergies, user lifestyle and user diet.

12. (canceled)

13. A non-transitory computer-readable storage medium having stored therein instructions which, when executed by a processor, cause the processor to perform operations comprising:
creating a user profile based at least upon received user genetic information;
storing the user profile in memory on a computer;
updating the user profile based on received user personal information, the received personal information including any medications currently used by the user;
storing information in a medication-interaction database, the information in the medical-interaction database obtained from at least one medical reference source;
updating the stored information in the medication-interaction database to include current scientific and medical data changes as they become available;
comparing information in the updated user profile to the updated information in the medication-interaction database and
presenting on a user's computing device, a visual display indicating if there are any interactions between a medication to be taken by the user and the user's genetic makeup, other medications taken by the user, the user's current illnesses, the user's lifestyle, the user's diet, and the user's allergies.

14. The non-transitory computer-readable storage medium of claim 13, wherein the user genetic information is obtained from a pharmacogenetic test.

15. (canceled)

16. The non-transitory computer-readable storage medium of claim 13, wherein the received genetic information contains single nucleotide polymorphisms (SNPs) for the user.

17. The non-transitory computer-readable storage medium of claim 13, wherein the user personal information includes at least one of user illnesses, user allergies, user lifestyle and user diet.

18. (canceled)

19. A personalized medication management and alert system, the system comprising:
means for obtaining a sample from a user, the sample containing user genetic information, the user genetic information containing single nucleotide polymorphisms (SNPs) for the user; and
a data management system configured to:
create a user profile based at least upon the user genetic information contained in the user sample;
store the user profile in memory on a computer;
update the user profile based on received user personal information, the received personal information including any medications currently used by the user and at least one of user illnesses, user allergies, user lifestyle and user diet;
store information in a medication-interaction database, the information in the medical-interaction database obtained from at least one medical reference source;
update the stored information in the medication-interaction database to include current scientific and medical data changes as they become available;
compare, by a processor, information in the updated user profile to the updated information in the medication-interaction database
present on a user's computing device, a visual display indicating if there are any interactions between a medication to be taken by the user and the user's genetic makeup, other medications taken by the user, the user's current illnesses, the user's lifestyle, the user's diet, and the user's allergies, and if there are interactions further displaying a severity of the interaction.

20. The method of claim 1, wherein the medication-interaction database includes information other than from previous patient interactions.

21. The method of claim 1, further comprising presenting on the user's computing device a severity of each interaction.

22. The method of claim 1, wherein the visual display presents interactions on a single display screen of the user's computing device.
